(19) United States
(12) Patent Application Publication
(10) Pub. No.: US 2021/0302318 A1
(43) Pub. Date: Sep. 30, 2021

(54) MEANS AND METHOD FOR THE REAL-TIME ANALYSIS OF HARVESTED CROPS

(71) Applicant: THE STATE OF ISRAEL, MINISTRY OF AGRICULTURE & RURAL DEVELOPMENT, AGRUCULTURAL RESEARCH ORGANIZATION, Rishon Lezion (IL)

(72) Inventor: Evgeni ELZTOV, Ashdod (IL)

(21) Appl. No.: 17/260,483

(22) PCT Filed: Jul. 16, 2019
(86) PCT No.: PCT/IL2019/050797

§ 371 Date: Jan. 14, 2021

Related U.S. Application Data
(60) Provisional application No. 62/698,307, filed on Jul. 16, 2018.

(51) Int. Cl. G01N 21/76; G01N 33/53

(52) U.S. Cl. CPC G01N 21/763; G01N 2201/0221; G01N 33/5308

(57) ABSTRACT

The invention provides a biosensor for the real-time analysis of Volatile Organic Compounds (VOC), comprising a bio-receptor cell sensitive to VOC's released from harvested crops, insects and microorganisms, a photodetector for detecting bioluminescence of the bio-receptor cell and converting it to a signal, and a transmitter for receiving and outputting data from said photodetector to a data processing module, wherein the data processing module comprises a rules based algorithm for differentiating between VOC profiles produced by ripening and spoiling of harvested crops.

### FIELD OF THE INVENTION

The field of the invention relates to means and methods for analysis of harvested crops and, more particularly, to biosensors for the real-time analysis of volatile organic compounds.

### BACKGROUND OF THE INVENTION

The problem of food loss post-harvest and throughout the supply chain is a major problem and a threat to nutricianal security. Worldwide, post-harvest losses is estimated at up to 50% of the harvested crop, said loss mainly due to rot caused by microorganisms. Current monitoring technology does not provide real-time and continuous information about precise location and timing the development of decay at the carton and or pallet level. To mitigate these losses, there is an increasing demand for efficient real-time monitoring of decay development in fruit and vegetables after harvest and through supply chain.

The present invention proposes a sensor that integrates whole cell bioreporters with commercially available active pixel sensor probes to provide a simple, portable and cost efficient solution, and to provide real-time, localized and continuous monitoring of crop quality during all post-harvest stages.

### SUMMARY OF THE INVENTION

It is an object of the present invention to provide a biosensor for the real-time analysis of Volatile Organic Compounds (VOC), wherein said biosensor comprising
a. at least one bio-receptor cell sensitive to VOC's released from harvested crops, insects and microorganisms;
b. at least one photodetector for detecting bioluminescence of said bio-receptor cell and converting to a signal, and
c. at least one transmitter for receiving and outputting data from said photodetector to a data processing module
   wherein said data processing module comprises a rules-based algorithm for differentiating between VOC profiles produced by ripening and spoiling of harvested crops.

It is another object of the present invention to provide the aforementioned biosensor, wherein the bio-receptor cell comprises whole cell bio-receptors or immobilized bioluminescent bacteria.

It is another object of the present invention to provide the aforementioned sensor provided with immobilization means for cells, selected from the group comprising hydrogels, synthetic membranes, absorption, covalent, sol-gel and any other conventional means of immobilization.

It is another object of the present invention to provide the aforementioned biosensor, wherein said bio-receptor cell is calibrated to sense to volatile organic compounds produced during ripening/spoilage of harvested crops (fruit, vegetables, tubers, grains) by said crops, by insects or by microorganisms or by cold damage or transportation damage.

It is another object of the present invention to provide the aforementioned biosensor, wherein said VOCs are biomarkers of crop ripening or spoilage selected from but not limited to, a group consisting of Aldehydes, Alcohols, Essential oils and Isothiocyanates.

It is another object of the present invention to provide the aforementioned biosensor, wherein said VOCs are biomarkers of crop ripening or spoilage selected from but not limited to, a group consisting of Limonene, α-Pinene, 2E-hexenal, Benzaldehydes, Acetic acid, ethanol, butyl acetate, ethyl acetate and ethanol etc.

It is another object of the present invention to provide the aforementioned biosensor, wherein biosensor includes additional sensors for analyzing environmental conditions, said environmental conditions selected from, but not limited to, a group consisting of temperature, humidity, barometric pressure, visible and UV light, infra-red, or any combination of.

It is another object of the present invention to provide the aforementioned biosensor, wherein the biosensor includes a data logger that stores all data generated by the biosensor.

It is another object of the present invention to provide the aforementioned biosensor, wherein the photodetector is an active-pixel sensor (APS).

It is another object of the present invention to provide the aforementioned biosensor, wherein the APS is constructed using integrated circuit technology, said technologies selected from a group consenting of; charge-coupled device (CCD), complementary metal-oxide-semiconductor (CMOS), Quanta Image Sensor, N-type metal-oxide-semiconductor logic (NMOS, live MOS).

It is another object of the present invention to provide the aforementioned biosensor, wherein the transmitter communicates using wired or wireless technologies selected from a group consisting of, but not limited to, wireless local area networks (WLAN), Low-Power Wide-Area Networks (LPWAN), wireless wide area network (WWAN), wireless sensor networks, Bluetooth, cellular networks, and any combination of.

It is another object of the present invention to provide the aforementioned biosensor, wherein the sensor is powered by an internal electricity source, said source being a battery.

It is an object of the present invention to disclose a method for the real-time monitoring of Volatile Organic Compounds comprising:
a. placing one or more biosensor in a storage area,
b. connecting the biosensor to a wired or wireless communications network,
c. connecting the network to a computer readable medium (CRM), capable of receiving and presenting the real-time VOC profile.
   It is another object of the present invention to provide a method, wherein said biosensor contains a bio-receptor cell, said bio-receptor cell is calibrated to sense volatile organic compounds produced during ripening/spoilage of harvested crops (fruit, vegetables, tubers, grains) by said crops, by insects or by microorganisms by cold damage or transportation damage.

It is another object of the present invention to provide a method, wherein the sensor network includes additional sensors for monitoring environmental conditions, said environmental conditions selected from, but not limited to, a group consisting of; temperature, humidity, barometric pressure, visible and UV light, infra-red, or any combination of.

It is another object of the present invention to provide a method, wherein the network is configured to operate using wired or wireless technologies selected from a group consisting of, but not limited to, wireless local area networks (WLAN), Low-Power Wide-Area Networks (LPWAN), wireless wide area network (WWAN), wireless sensor networks, Bluetooth, cellular networks, and any combination thereof .s

It is another aspect of the invention to use cells immobilization approaches, e.g., hydrogels, synthetic membranes, absorption, covalent, sol-gel and any other conventional s method.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings, in which

FIG. 1 is a schematic representation of an embodiment of the present invention;

FIG. 2 is a schematic representation of a method of the present invention;

FIGS. 3A and 3B shows preliminary results of the present invention;

FIG. 4 shows preliminary results of the present invention;

FIG. 5 shows preliminary results of the present invention;

FIG. 6 shows preliminary results of the present invention;

FIG. 7 shows preliminary results of the present invention.

FIGS. 8A to 8D shows preliminary results of the present invention;

FIG. 9 shows preliminary results of the present invention;

FIG. 10 shows preliminary results of the present invention;

FIG. 11 shows preliminary results of the present invention;

FIG. 12 shows preliminary results of the present invention;

FIG. 13 shows preliminary results of the present invention;

FIG. 14 shows preliminary results of the present invention;

FIG. 15 shows preliminary results of the present invention; and

FIG. 16 shows preliminary results of the present invention;

### DETAILED DESCRIPTION OF THE INVENTION

The following description is provided, alongside all chapters of the present invention, so as to enable any person skilled in the art to make use of said invention and sets forth the best modes contemplated by the inventor of carrying out this invention. Various modifications, however, are adapted to remain apparent to those skilled in the art, since the generic principles of the present invention have been defined specifically to provide a biosensor for the real-time analysis of volatile organic compounds and method of using the same.

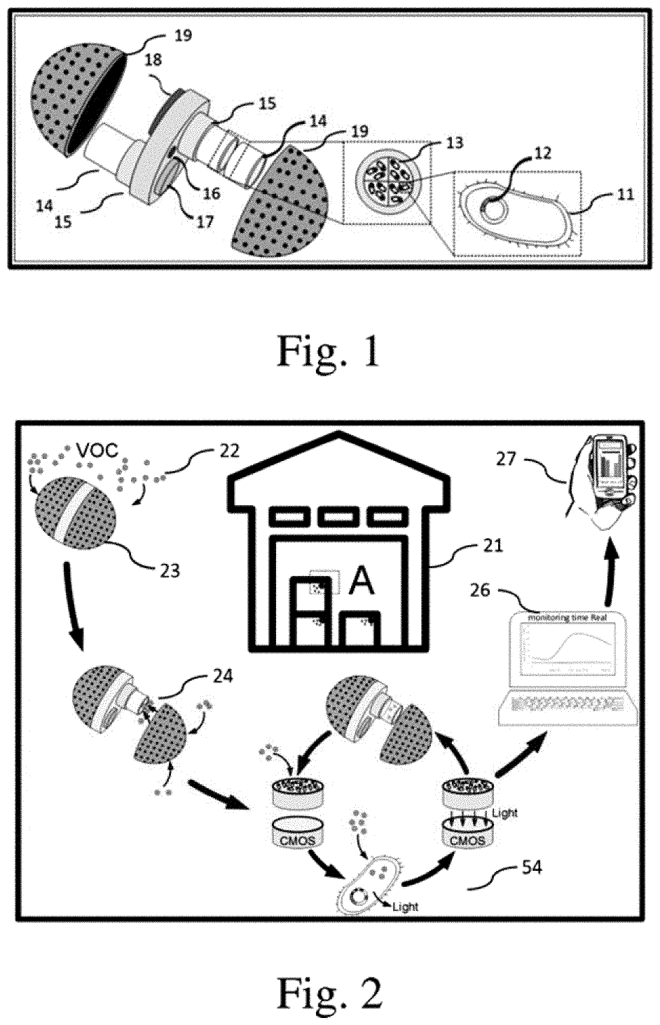

FIG. 1 shows the schematic of the proposed structure of the current invention.

FIG. 2 is a schematic representation of a method of the present invention.

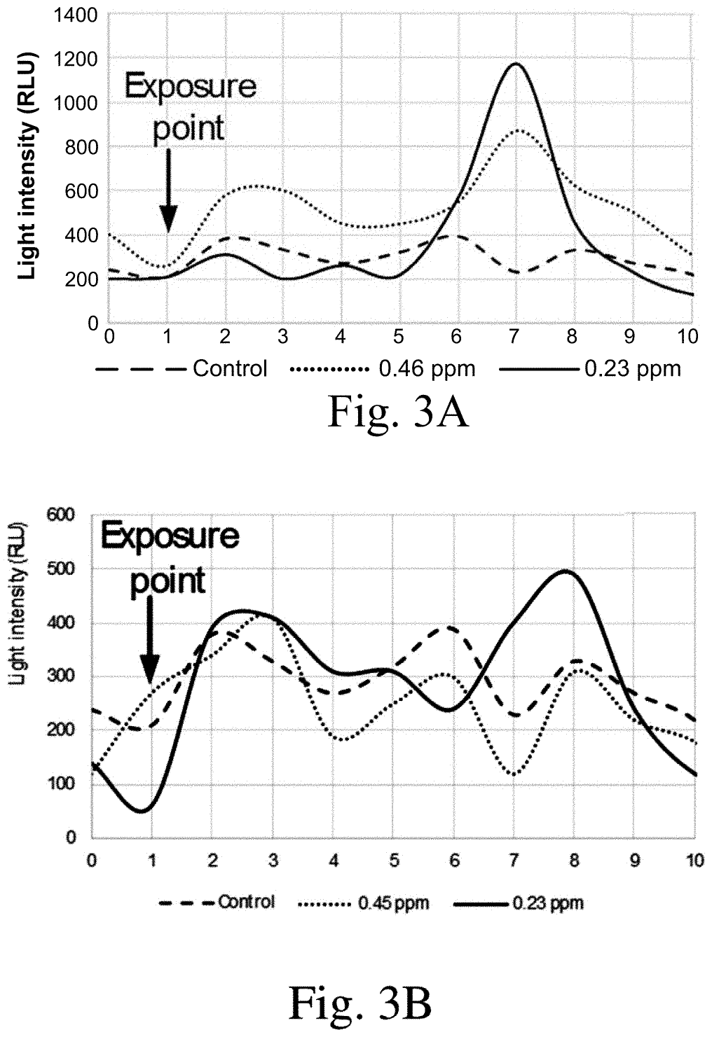

FIGS. 3A and 3B show the kinetic response of the TV1061 strains to the different concentrations of (A) Limonene (B) α-Pinene.

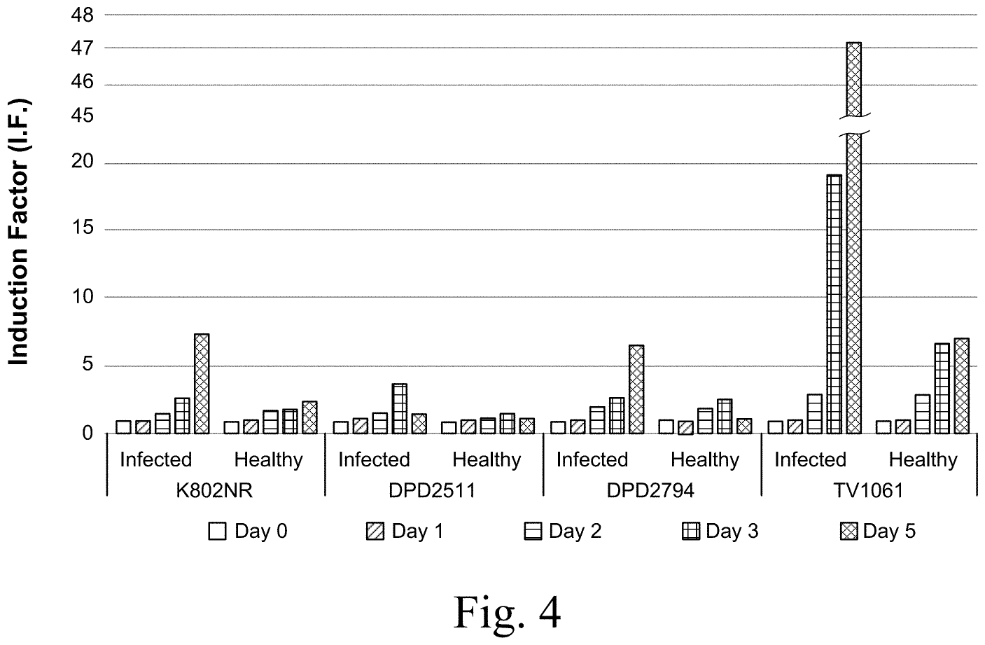

FIG. 4 shows the kinetic response of the various bioluminescent strains (K802NR-quorum sensing, DPD2511-oxidation, DPD2794-genotoxic, TV1061-cytotoxic) to the infected and un-infected oranges.

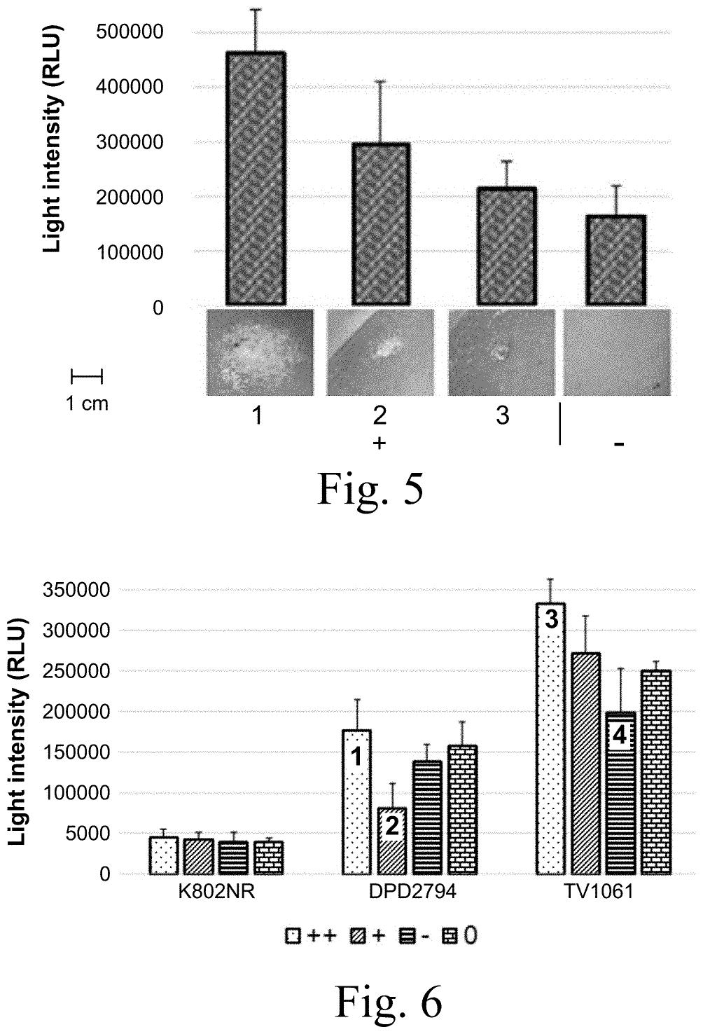

FIG. 5 shows the effect of the infection progression on the signal values.

FIG. 6 shows the reaction of bioluminescence bacteria (TV1061, DPD2794 and NR802K) to the VOC released from infected (+) and non-infected (−) wheat.

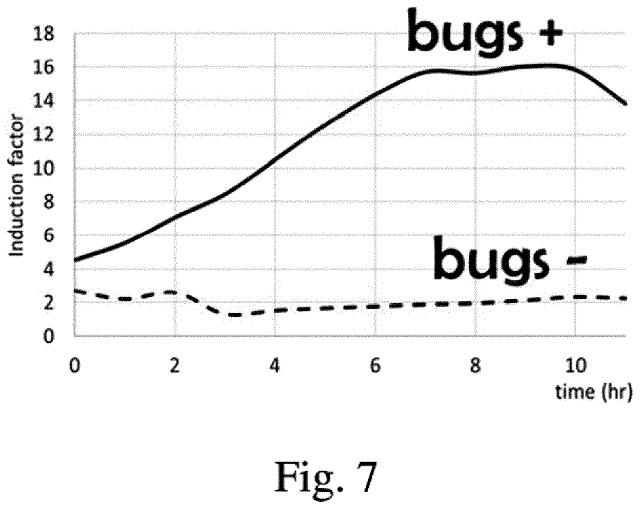

FIG. 7 shows real time monitoring the presents of insects in harvested wheat.

The object of this invention is a biosensor for the real-time tracking of harvested crops.

The immobilized bioluminescent bacteria in these sensors will monitor the VOCs profile in the air, and a signal change will be generated reflecting the status of crops health or disease. Coupled with the CMOS sensor, these signals are transmitted and translated to a smartphone application for the end-user ease of usage. FIG. 1 shows a non-limiting embodiment of a sensor that combines a biological-sensor cell with a photodetector. The biosensor cell 13, 14 contains immobilized bioluminescent bacteria 11 that have been genetically engineered 12 to exhibit increased sensitivity to volatile organic compounds (VOC) released by crops following harvest, by insects or by microorganism (such as fungi, yeast, bacteria etc.). The bioluminescent bacteria react to VOC by emitting luminescence that is translated by the photodetector 15 to a signal that can be transmitted to data processing module. Using complementary metal-oxide semiconductor (CMOS) technology to construct the active-pixel sensor (APS) photodetector enables the production of a more cost efficient sensor and therefore the potential for the deployment of more units for better coverage. Alternative technologies, such as charge-coupled device (CCD) technology can also be used for the construction of the active-pixel sensor (APS), as CCD grants the sensor greater sensitivity and faster response time.

The photodetector 15 and temperature sensor 16 are connected to a transmitter 18 and the whole system is power by an internal electricity source 17. The biosensor is encapsulated in a permeable sensor cap 19 that enables VOC to pass through. The sensor is configured to enable the incorporation of additional sensing units capable of tracking various environmental conditions (such as a humidity, barometric pressure, light, IR and UV exposure, etc.) during storage and transport.

FIG. 2 shows one non-limiting embodiment of the storage tracking system. The biosensors are strategically placed in the storage area 21. The VOCs 22 spread throughout the storage area and enter the biosensor through the permeable cap 23 to reach the biological sensor-cell 24. The VOC interact with the bioluminescent bacteria and activate the photodetector 25. The data in transmitted to data processing module and display units, such as programs installed on computer terminals 26 and cellular phones 27. The processing module converts the data, differentiates the VOC's profile and displays the real-time VOC analysis.

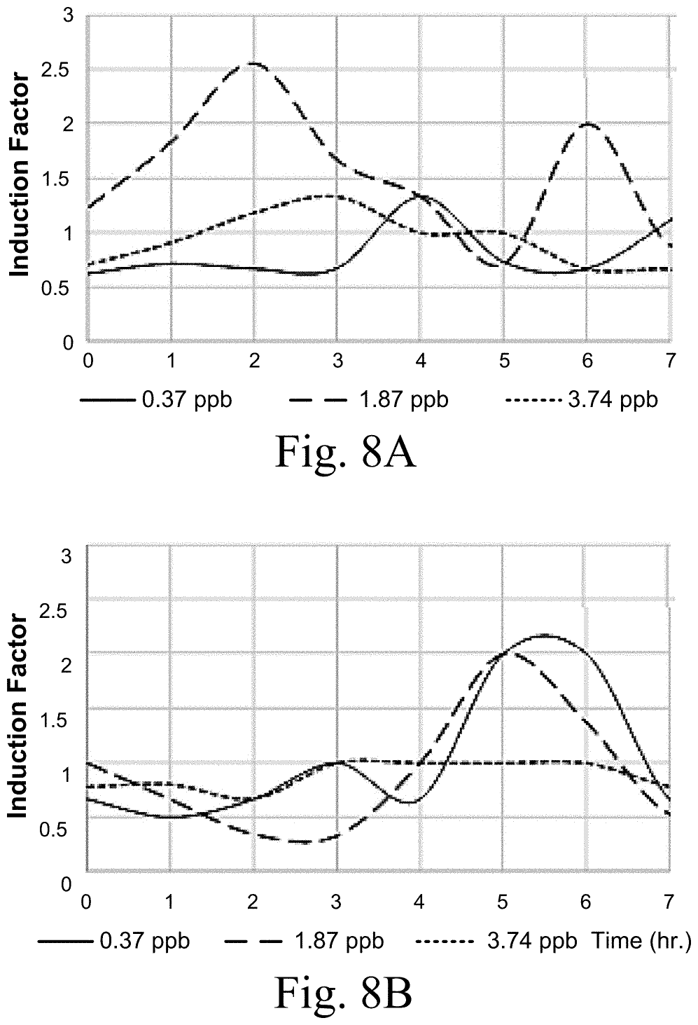

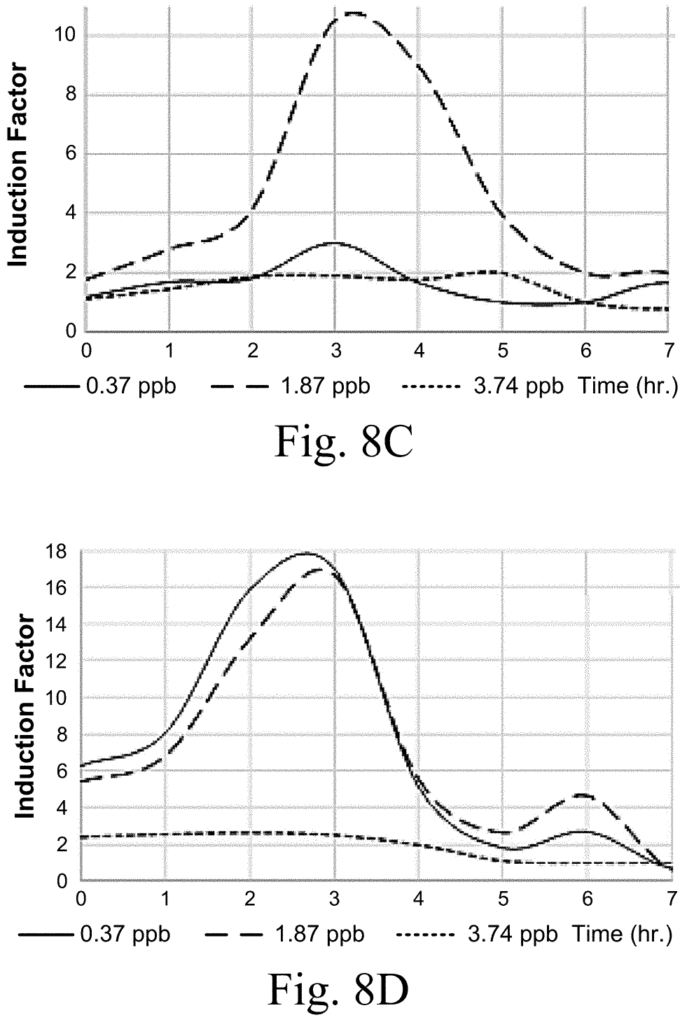

Reference is now made to FIGS. 8A to 8D demonstrating the effect on the four different bioluminescent strains, sensitive to the various stresses. The four strains tested responded differently to the limonene. Nevertheless that all bacterial strains were induced by limonene presence in the air, the highest response was observed with TV1061 strain, cells sensitive to the cytotoxic stresses (FIG. 8D). Such responses enforced this assumption for the possible limonene cytotoxicity.

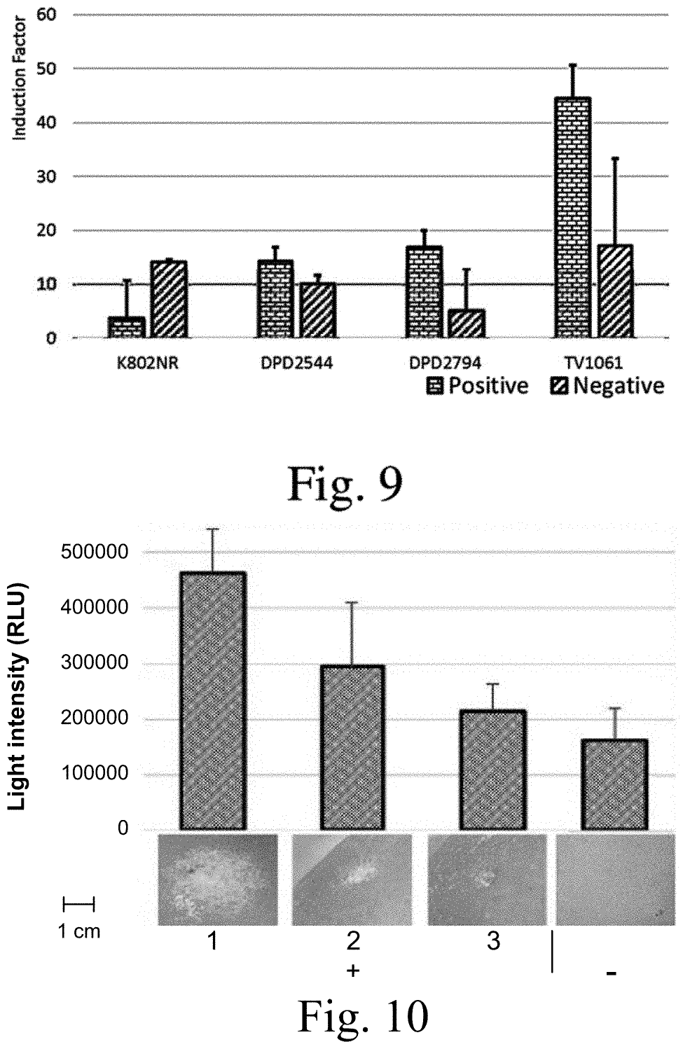

Reference is now made to FIG. 9 determining the capability of the bioreporter bacteria. VOCs emitted from oranges infected with P. digitatum was sensed, The bioreporter bacteria enclosed in calcium alginate beads were exposed to infected fruit at different stages of decay development in sealed glass jar containers. FIG. 9 show the response of these bacterial strains to the VOCs produced by infected and uninfected samples. Similar to the responses obtained with the pure limonene, the highest difference between infected and uninfected fruits were observed with strain TV1061, sensitive to cytotoxic stresses.

Reference is now made to FIG. 10 demonstrating the effect of the infection stage on sensor responses. TV1061 strain was exposed to infected fruit at different times after infection. It can be observed that infection progress increased bioluminescent output responses.

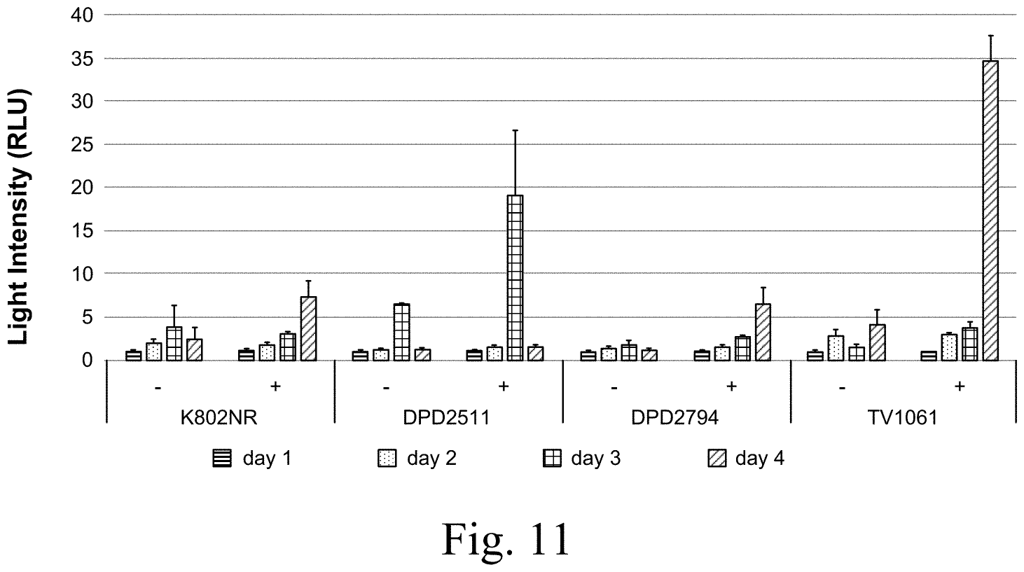

Reference is now made to FIG. 11 showing kinetic response of the various bioluminescent strains (K802NR-quorum sensing, DPD2511-oxidation, DPD2794-genotoxic, TV1061-cytotoxic) to the Penicillium Digitatum infected and un-infected oranges. To check the capability of the proposed system to detect infection, air near infected and uninfected fruits were monitored with four different bacterial strains (FIG. 11). As in the case with limonene, each used strain responded differently to the infected fruits, while proposed biosensor detected problems at the third day from the infection point, before any visible fungus marks on the orange surface. Furthermore, increase in the bacterial responses with disease progression, are pointing to a correlation between infection state and its effect on cells luminescence (FIG. 11). Indeed, the strongest VOCs effect on bacteria was observed in the last measurement point, a day with the highest VOCs differences between infected and uninfected samples.

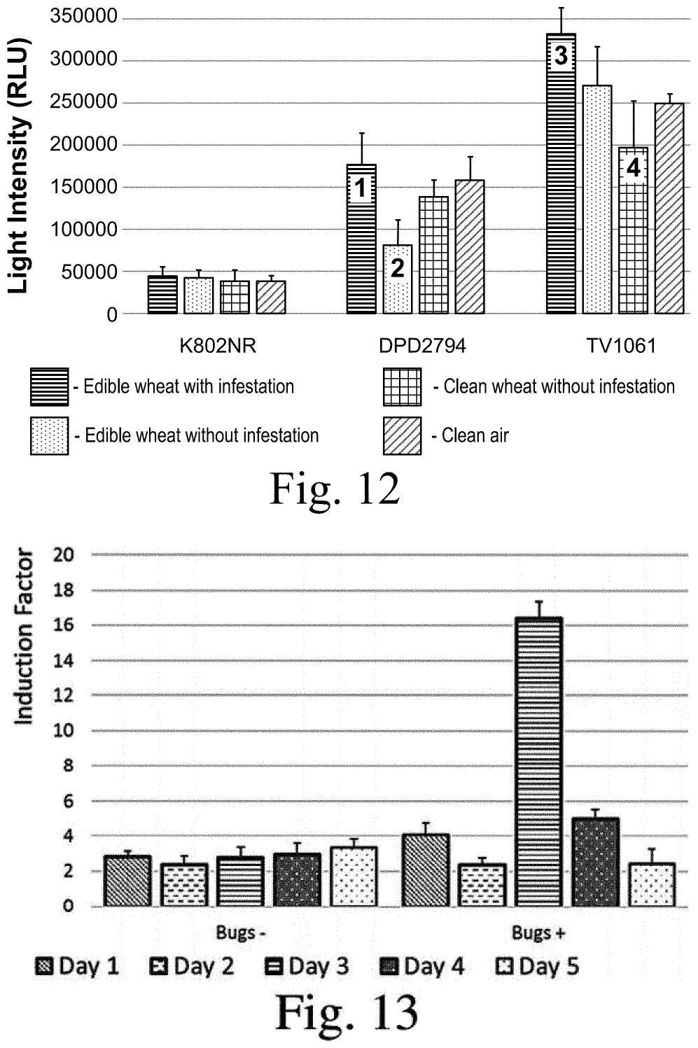

Reference is now made to FIG. 12 showing a response of different bioreporter bacterial strains (K802NR-quorum sensing, DPD2794-genotoxic, TV1061-cytotoxic) to the insects contaminated wheat. To check the capability of the proposed system to detect the presence of the rise weevil in wheat, air near damaged wheat with and without insects, uninfected wheat and empty chamber were monitored with three different bacterial strains. Each strain responded differently to each sample, but it is clearly may be observed the difference between damaged and undamaged wheat.

Reference is now made to FIG. 13 showing monitoring efficiency of Deltamethrin pest control treatment in wheat. To determinate system capability to test the efficiency of the pest control treatments, infected and clean wheat were treated with Deltamethrin. Then, sensor (with TV1061 strain) was placed into the treated chambers. Similar responses in the infected and uninfected sample are indicating that all insects were destroyed during the sanitization process (FIG. 13). Deltamethrin is not effective against eggs inside the wheat. Therefore insects inside wheat (in eggs) will be destroyed at the time when they will hatch and walk out. Indeed FIG. 13 demonstrated an increase in the bacteria responses on the third day after the sanitization process, the date when undamaged insects came out from the wheat. Then decreasing in the sensor responses to the background levels may be explained by killing these insects by Deltamethrin residues on the wheat.

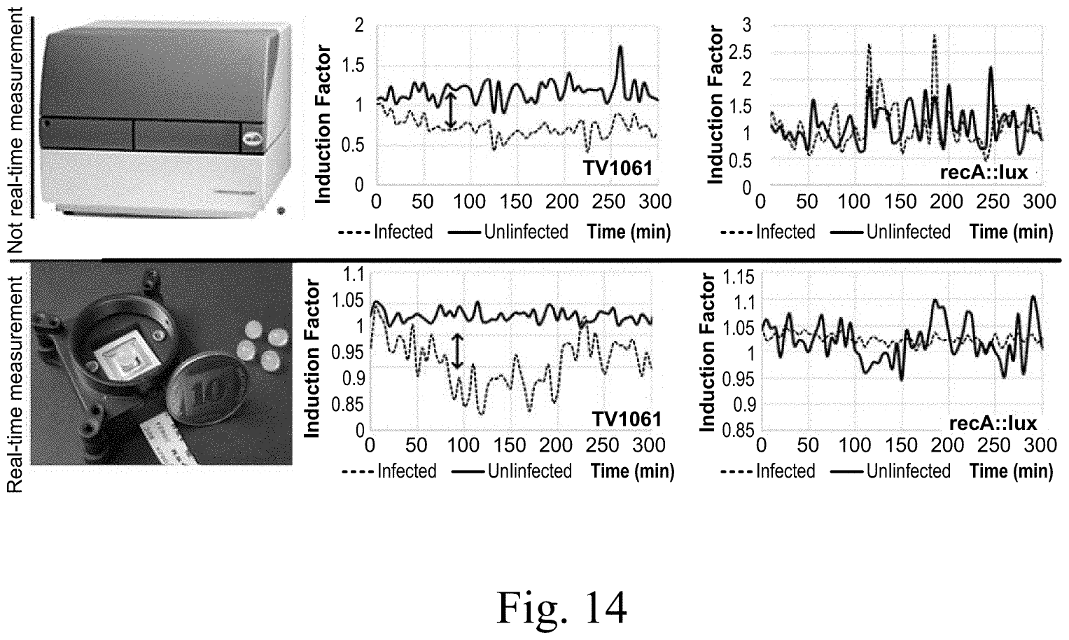

Reference is now made to FIG. 14 showing responses of the cytotoxic (TV1061) and genotoxic (rec::lux) strains to the infection in potato. Bacteria were measured by commercial available luminometer and CMOS based application. FIG. 14 demonstrates the capability of the CMOS based biosensor to detect presence infection in the potato tubers. The response pattern of the CMOS based application for both tested strains was similar to a commercially available device.

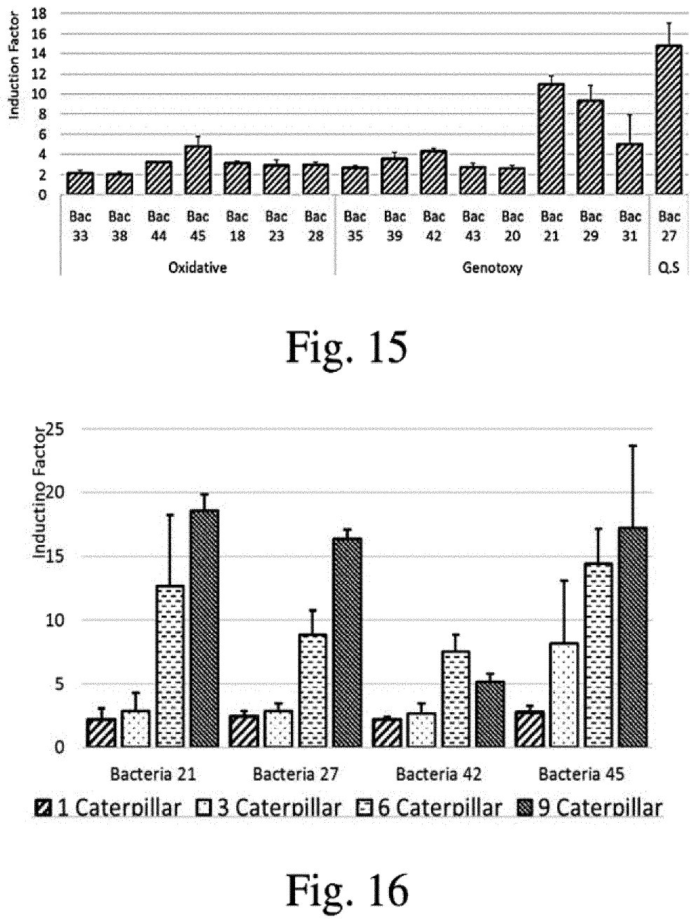

Reference is now made to FIG. 15 showing response of the different quorum sensing, oxidative and genotoxic strains to the false codling moth caterpillars presence in the oranges. FIG. 15 demonstrates the response of the 16 different strains to the false codling moth caterpillars. As it may be observed different strains responded differently, but high response values of the strains 21, 29, 31 and 27 suggesting not only their sensitivity to the presence of false codling moth caterpillars but also capability to use them in the future CMOS application.

Reference is now made to FIG. 16 showing the dose depended on responses to the false codling moth caterpillars. To determine the dose dependence capability of the proposed system, four strains immobilized in calcium alginate hydrogel were exposed to different false codling moth caterpillars concentrations. It can be observed that with the increase in the caterpillar numbers increased bioluminescent responses.

1. A biosensor for the real-time analysis of Volatile Organic Compounds (VOC), said biosensor comprising
a. at least one bio-receptor cell sensitive to VOC's released from harvested crops, insects and microorganisms;
b. at least one photodetector for detecting bioluminescence of said bio-receptor cell and converting to a signal, and
c. at least one transmitter for receiving and outputting data from said photodetector to a data processing module
wherein said data processing module comprises a rules-based algorithm for differentiating between VOC profiles produced by ripening and spoiling of harvested crops.

2. The Biosensor of claim 1, wherein the bio-receptor cell comprises whole cell bio-receptors or immobilized bioluminescent bacteria.

3. The Biosensor of claim 2, wherein said bio-receptor cell is calibrated to sense to volatile organic compounds produced during ripening/spoilage of harvested crops (fruit, vegetables, tubers, grains) by said crops, by insects , by microorganisms cold or transportation damage.

4. The Biosensor of claim 3, wherein said VOCs are biomarkers of crop ripening or spoilage selected from but not limited to, a group consisting of Aldehydes, Alcohols, Essential oils and Isothiocyanates.

5. The Biosensor of claim 3, wherein said VOCs are biomarkers of crop ripening or spoilage selected from but not limited to, a group consisting Limonene, α-Pinene, 2E-hexenal, Benzaldehydes, Acetic acid, ethanol, butyl acetate, ethyl acetate and ethanol etc.

6. The Biosensor of claim 1, wherein biosensor includes additional sensors for analyzing environmental conditions, said environmental conditions selected from, but not limited to, a group consisting of; temperature, humidity, barometric pressure, visible and UV light, infra-red, or any combination of.

7. The Biosensor of claim 1, wherein the biosensor includes a data logger that stores all data generated by the biosensor.

8. The Biosensor of claim 1, wherein the photodetector is an active-pixel sensor (APS).

9. The Biosensor of claim 7, wherein the APS is constructed using integrated circuit technology, said technologies selected from a group consenting of charge-coupled device (CCD), complementary metal-oxide-semiconductor (CMOS), Quanta Image Sensor, N-type metal-oxide-semiconductor logic (NMOS, live MOS).

10. The Biosensor of claim 1, wherein the transmitter communicates using wired or wireless technologies selected from a group consisting of, but not limited to, wireless local area networks (WLAN), Low-Power Wide-Area Networks (LPWAN), wireless wide area network (WWAN), wireless sensor networks, Bluetooth, cellular networks, and any combination of.

11. The Biosensor of claim 1, wherein the sensor is powered by an internal electricity source, said source being a battery.

12. The biosensor according to claim 1 wherein said sensor is provided with cells immobilization means selected from the group comprising hydrogels, synthetic membranes, absorption, covalent, sol-gel and any other conventional means of immobilization.

13. A method for the real-time monitoring of Volatile Organic Compounds (VOC) comprising:
a. placing one or more biosensor in a storage area,
b. connecting the biosensor to a wired or wireless communications network,
c. connecting the network to a computer readable medium (CRM), capable of receiving and presenting the real-time VOC profile.

14. The method of claim 13, wherein said biosensor contains a bio-receptor cell, said bio-receptor cell is calibrated to sense volatile organic compounds produced during ripening and spoilage of harvested crops (fruit, vegetables, tubers, grains) by said crops, by insects or by microorganisms or by cold damage or transportation damage.

15. The method of claim 13, wherein the sensor network includes additional sensors for monitoring environmental conditions, said environmental conditions selected from, but not limited to, a group consisting of; temperature, humidity, barometric pressure, visible and UV light, infra-red, or any combination of.

16. The method of claim 13, wherein the network is configured to operate using wired or wireless technologies selected from a group consisting of, but not limited to, wireless local area networks (WLAN), Low-Power Wide-Area Networks (LPWAN), wireless wide area network (WWAN), wireless sensor networks, Bluetooth, cellular networks, and any combination of.
